(19) United States
(12) Patent Application Publication
(10) Pub. No.: US 2017/0080181 A1
(43) Pub. Date: Mar. 23, 2017

(54) CATHETER, CATHETER PRODUCTION MOLD, CATHETER PRODUCTION METHOD

(71) Applicant: OLYMPUS CORPORATION, Tokyo (JP)

(72) Inventors: Junji SHIONO, Yokohama-shi (JP); Yusuke NOMURA, Yokohama-shi (JP)

(73) Assignee: OLYMPUS CORPORATION, Tokyo (JP)

(21) Appl. No.: 15/370,096

(22) Filed: Dec. 6, 2016

Related U.S. Application Data
(63) Continuation of application No. PCT/JP2015/063747, filed on May 13, 2015.

(30) Foreign Application Priority Data
Jun. 27, 2014 (JP) 2014-133187

(51) Int. Cl. A61M 25/00; B29C 45/26; A61M 25/10

(52) U.S. Cl. CPC A61M 25/0026; A61M 25/0009; A61M 25/10; B29C 45/261; A61M 2025/0037; A61M 2025/0039; B29L 2031/7542

(57) ABSTRACT

A catheter includes: a conduit part having a plurality of lumens whose external diameter at a tip side thereof is constant, each of the plurality of lumens extending along an axis line inside the conduit part; and a branched part that separates axis lines of the plurality of lumens from each other at a base end side of the conduit part. At an end of the conduit part inserted into inside the branched part, an opening of each of the plurality of lumens is provided along a different axis line with each other, and a level difference is formed at the end of the conduit part.

### CROSS REFERENCE TO RELATED APPLICATIONS

This application is a continuation application based on a PCT Patent Application No. PCT/JP2015/063747, filed on May 13, 2015, whose priority is claimed on Japanese Patent Application No. 2014-133187, filed on Jun. 27, 2014, the entire content of which are hereby incorporated by reference.

### BACKGROUND OF THE INVENTION

Technical Field

The present invention relates to a catheter, a catheter production mold, and a catheter production method.

Description of the Related Art

Treatment tools that perform treatment on a living body include catheters used for endoscopes or the like (for example, refer to Japanese Unexamined Patent Application, First Publication No. 2005-323739, Japanese Patent No. 2680067, and Japanese Examined Patent Application, Second Publication No. H6-59314). These catheters have, for example, a plurality of lumens, and have a plurality of conduits substantially over the entire length thereof. Among these catheters, in a balloon catheter or the like, at least some of the plurality of conduits are blocked or sealed at one end. Additionally, a catheter in which both ends of a conduit are open is also used.

In a medical balloon catheter according to Japanese Examined Patent Application, Second Publication No. H6-59314, a balloon is attached to a tip of a flexible tube, and a main passage for delivering medicines, such as a contrast medium, and a passage that supplies a fluid for inflating the balloon are integrally formed in the flexible tube. Additionally, the flexible tube branches into two tubes individually connected to the main passage and the passage by a branched part provided on a base end side. Separate mouthpieces are respectively attached to extending tips of the two tubes.

In such catheters, the main passage and the passage branch at a proper angle with respect to a tube axis direction in the branched part, the surroundings of the passages are covered with resin, such as an adhesive, and this resin is solidified in a suitable shape to form the branched part.

### SUMMARY

A catheter of a first aspect of the present invention includes: a conduit part having a plurality of lumens whose external diameter at a tip side thereof is constant, each of the plurality of lumens extending along an axis line inside the conduit part; and a branched part that separates axis lines of the plurality of lumens from each other at a base end side of the conduit part, wherein, at an end of the conduit part inserted into inside the branched part, an opening of each of the plurality of lumens is provided along a different axis line with each other, and a level difference is formed at the end of the conduit part.

According to a second aspect of the present invention, in the above first aspect, an inner tube in which at least one lumen of the plurality of lumens are formed may be provided at inside of the conduit part, and an end of the inner tube may be extended toward a base end side of the branched part than the end of the conduit part inserted into inside the branched part.

According to a third aspect of the present invention, in the above second aspect, the inner tube may be provided only around inside of the branched part.

According to a fourth aspect of the present invention, in the above first aspect, an inner tube in which at least one lumen of the plurality of lumens are formed may be provided at inside of the conduit part, the inner tube may be provided as a coaxial lumen inside an outer tube of the conduit part, and an end of the inner tube may be extended toward a base end side of the branched part than an end of the outer tube inserted into inside the branched part.

According to a catheter production mold of a fifth aspect of the present invention, there is provided a catheter production mold for producing a catheter including: a conduit part having a plurality of lumens whose external diameter at a tip side thereof is constant, each of the plurality of lumens extending along an axis line inside the conduit part; and a branched part that separates axis lines of the plurality of lumens from each other at a base end side of the conduit part, wherein an inner tube is provided as the lumen at inside of the conduit part, the inner tube is provided as a coaxial lumen inside an outer tube of the conduit part, and an end of the inner tube is extended toward a base end side of the branched part than an end of the outer tube inserted into inside the branched part. The mold includes a first core bar inserted into a gap between the outer tube and the inner tube located within the outer tube; a second core bar inserted into the inner tube; and an outer mold that covers the first core bar and the second core bar to form an external form of the branched part. In the first core bar, an insertion part to be inserted into the outer tube has an outer surface part along the inside of the outer tube, and an inner surface part along the outside of the inner tube, the inner surface part is a groove corresponding to an external form of the internal tube, and a termination end is provided in the groove of the insertion part in a length direction of the groove, wherein the first core bar increases in diameter from the termination end toward a base end side of the branched part. Groove parts that allow the outer tube, the inner tube, and the first and second core bars inserted into the outer and inner tubes to be disposed therein and branch into a Y shape, and a space part that allows the branched part to be formed therein together with the groove parts are disposed in the outer mold.

According to a sixth aspect of the present invention, in the above fifth aspect, a sectional shape of the insertion part of the first core bar in a direction intersecting an axis is substantially a crescent. Additionally, a sectional shape of the second core bar in a direction intersecting an axis is substantially circular.

A catheter production method of a seventh aspect of the present invention is a method for producing a catheter using the above mold. The method includes a step of inserting the second core bar into the inner tube; a step of disposing the inner tube, into which the second core bar is inserted, in the groove of the first core bar; a step of inserting the second core bar, the inner tube, and the first core bar into the outer tube; a step of disposing the second core bar, the inner tube, the first core bar, and the outer tube in the corresponding groove parts of the outer mold; a step of injecting resin into the space part to form the branched part; and a step of releasing the mold and removing the core bars.

### BRIEF DESCRIPTION OF THE DRAWINGS

FIG. 1 is a perspective view illustrating a first embodiment of a catheter related to the present invention.

FIG. 2 is an enlarged perspective view illustrating the vicinity of a branched part in the first embodiment of the catheter related to the present invention.

FIG. 3A is a sectional view illustrating the first embodiment of the catheter related to the present invention.

FIG. 3B is a sectional view illustrating the first embodiment of the catheter related to the present invention.

FIG. 3C is a sectional view illustrating the first embodiment of the catheter related to the present invention.

FIG. 3D is a sectional view illustrating the first embodiment of the catheter related to the present invention.

FIG. 4 is a plan view illustrating a first embodiment of a catheter production mold related to the present invention.

FIG. 5A is a perspective view illustrating the first embodiment of the catheter production mold related to the present invention.

FIG. 5B is a perspective view illustrating the first embodiment of the catheter production mold related to the present invention.

FIG. 5C is a sectional view illustrating the first embodiment of the catheter production mold related to the present invention.

FIG. 6 is a perspective view illustrating the first embodiment of the catheter production mold related to the present invention.

FIG. 7 is a flowchart illustrating a first embodiment of a catheter production method related to the present invention.

FIG. 8A is a process diagram illustrating a state before resin injection in the vicinity of a branched part in the first embodiment of the catheter production method related to the present invention.

FIG. 8B is a process diagram illustrating a state after resin injection in the vicinity of the branched part in the first embodiment of the catheter production method related to the present invention.

FIG. 9 is an enlarged perspective view illustrating a state before removal of core bar in the vicinity of the branched part in the first embodiment of the catheter production method related to the present invention.

FIG. 10 is a perspective view illustrating a second embodiment of a catheter related to the present invention.

FIG. 11A is an enlarged perspective view illustrating the vicinity of end of a conduit part in the second embodiment of the catheter related to the present invention.

FIG. 11B is an enlarged perspective view illustrating the vicinity of end of a conduit part in the second embodiment of the catheter related to the present invention.

FIG. 12 is a perspective view illustrating an example of the vicinity of end of a conduit part in the second embodiment of the catheter related to the present invention.

FIG. 13A is a perspective view illustrating an example of the vicinity of end of a conduit part in the second embodiment of the catheter related to the present invention.

FIG. 13B is a perspective view illustrating an example of the vicinity of end of a conduit part in the second embodiment of the catheter related to the present invention.

FIG. 14 is a perspective view illustrating an example of the vicinity of end of a conduit part in the second embodiment of the catheter related to the present invention.

FIG. 15 is a perspective view illustrating a third embodiment of a catheter related to the present invention.

### DETAILED DESCRIPTION OF THE PREFERRED EMBODIMENTS

Hereinafter, a first embodiment of a catheter, a catheter production mold, and a catheter production method related to the present invention will be described with reference to the drawings.

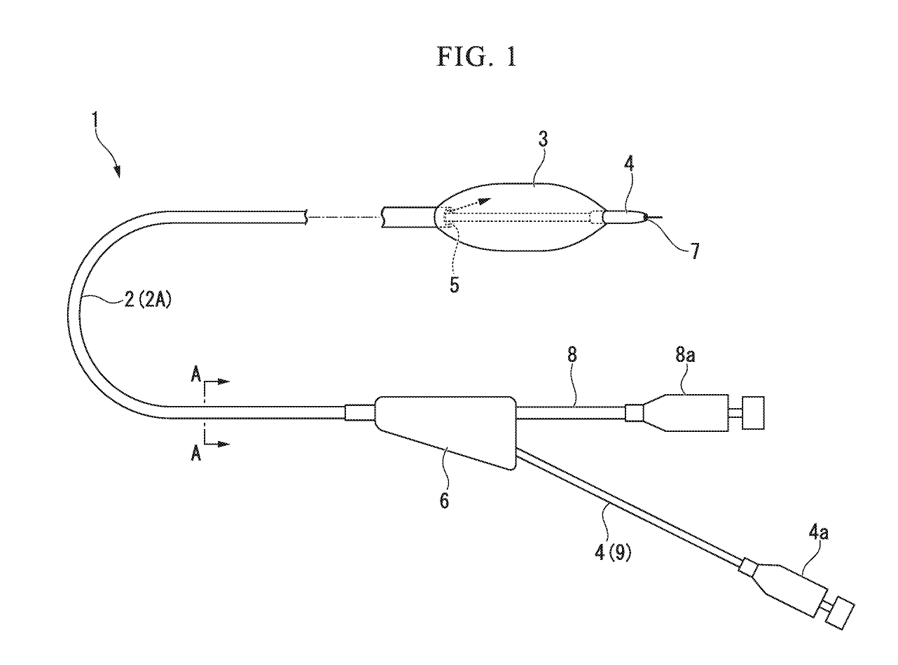

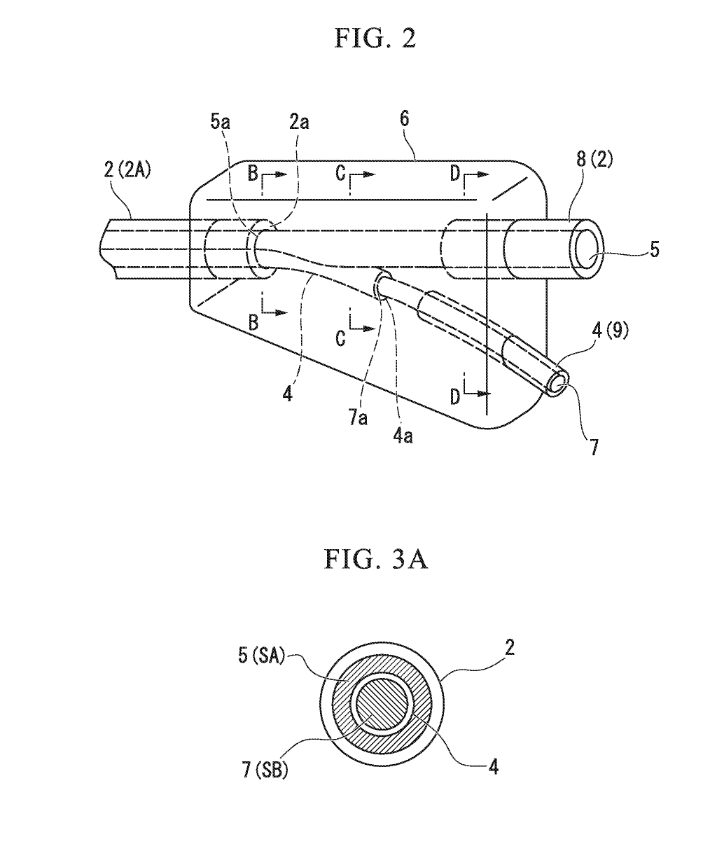

FIG. 1 is a perspective view illustrating a catheter in the present embodiment, FIG. 2 is an enlarged perspective view of FIG. 1, and reference sign 1 represents a catheter in the drawings.

The catheter 1 in the present embodiment is a balloon catheter. In addition, the present invention is not limited to the balloon catheter and can also be adapted to other types of catheters except for this balloon catheter.

As illustrated in FIG. 1, the catheter 1 in the present embodiment has a cylindrical outer tube 2 having flexibility, a balloon 3 attached to the tip of the outer tube 2, a cylindrical inner tube 4 provided coaxially (in a state where the central axis of the outer tube 2 and the central axis of the inner tube 4 coincide with each other) inside the outer tube 2, and a branched part 6 that separates the outer tube 2 and the inner tube 4 from each other on a base end (first end) side. The outer tube 2 and the inner tube 4 between the balloon 3 and the branched part 6 constitute a conduit part 2A.

In a portion closer to a tip side than the branched part 6, a first lumen 5 is formed by the inside of the outer tube 2 and the outside of the inner tube 4, and similarly, a second lumen 7 is formed within the inner tube 4.

Additionally, the outer tube 2 branches into a tube 8 individually connected to the first lumen 5 from the branched part 6 provided on the base end side, and a tube 9 that communicates with the second lumen 7. Separate mouthpieces 4a and 8b are respectively attached to extending tips of the two tubes 9 and 8.

Polyethylene resin or the like can be used for a catheter 1 in consideration of an adhesive property with the balloon 3 and workability. Additionally, a material that constitutes the outer tube 2 and the inner tube 4 may include, for example, vinyl chloride resin (particularly, soft vinyl chloride resin), silicone rubber, polyurethane resin, or the like.

Although the outer tube 2 and the inner tube 4 may be made of different materials, it is preferable that the outer tube and the inner tube are made of the same material. Accordingly, since the material of the medical catheter 1 is commonly used, productivity can be improved.

Although the internal diameter of the outer tube 2 is not particularly limited, 1 mm to 9 mm is preferable, and 2 mm to 4 mm is particularly preferable. Although the external diameter of the outer tube 2 is also not particularly limited, 2 mm to 10 mm is preferable, and 3 mm to 5 mm is particularly preferable. If the internal and external diameters of the inner tube are within the ranges, the outer tube 2 that is not bulky when the branched part is bundled and has also little flow rate resistance can be configured.

Although the internal diameter of the inner tube 4 is not particularly limited, 1 mm to 9 mm is preferable, and 0.3 mm to 3 mm is particularly preferable. Although the external diameter of the inner tube 4 is also not particularly limited, 1 mm to 5 mm is preferable, and 1 mm to 3 mm is particularly preferable. If the internal and external diameters of the inner tube are within the ranges, the inner tube 4 that is not bulky when being bundled and has also little flow rate can be configured.

The balloon 3 is a tube produced from, for example, polyethylene or nylon, and is provided at the tip of the catheter 1. The first lumen 5, for example, is configured so as to communicate up to the balloon 3 and supply a fluid for inflating the balloon 3.

The second lumen 7, for example, is open so as to communicate up to the tip side of the conduit part 2A of the catheter 1. When a tip part of the catheter 1 is guided into a lumen of an organ, a guide wire or a stylet can be inserted through the second lumen 7 or a contrast medium can be injected into the lumen.

The first lumen 5 that supplies a fluid to the balloon 3 does not reach a tip part of the inner tube 4 in the catheter 1. Additionally, in order to maintain the mechanical strength of this tip part, the second lumen 7 is located at the center. In order to improve insertability, the tip part of the inner tube 4 is configured so as to decrease in diameter in a tapered shape toward the tip and maintain the thickness of a peripheral edge of the tip over the entire circumference.

In the catheter 1, the outer tube 2 and the inner tube 4 are separated from each other in the branched part 6 such that a central axis of the outer tube 2 in a longitudinal direction and a central axis of the inner tube 4 in the longitudinal direction are separated from the tip part (second end) side in which the balloon 3 is provided toward the base end side. In addition, in the present embodiment, the outer tube 2 is fitted into the branched part 6 as a tube 8 that is a separate member that has the same external diameter as the external diameter of the outer tube 2, nearer the base end side than the branched part 6.

Additionally, the inner tube 4 is fitted into the branched part 6 as the a tube 9 that is a tube material that has the same external diameter as the external diameter of the inner tube 4, nearer the base end side than the branched part 6. The inner tube 4 may be a tube material that is continuous on the base end side and the tip side of the branched part 6.

In the branched part 6, an opening 5a of the first lumen 5 is formed in the end 2a of the outer tube 2, which is at the base end side of the conduit part 2A, inserted into the branched part 6. Also, an opening 7a of the second lumen 7 is formed in the end 4a of the inner tube 4, which is at the base end side of the conduit part 2A, inserted into the branched part 6. In the conduit part 2A, the end 2a and the end 4a form the level difference. In addition, although the first lumen 5 and the second lumen 7, which are channels for passing fluid, are not divided by the openings 5a and 7a, both of the outer tube 2 and the inner tube 4 inside the branched part 6 are tube materials and the openings 5a, 7a are formed in the ends 2a, 4a.

The opening 5a of the first lumen 5 and the opening 7a of the second lumen 7 are set to be along axis lines (in the directions of channels) different from each other, and the ends 2a and 4a of the outer tube 2 and the inner tube 4, which are at the base end side of the conduit part 2A, are formed.

Specifically, as shown in FIG. 2, the end 2a of the outer tube 2 is set to be in an area of 5 to 10 mm from the left side end of the branched part 6, and the end 4a of the inner tube 4 is set to be in an area of 10 to 20 mm from the left side end of the branched part 6.

That is, the length which the end 4a of the inner tube 4 extends from the end 2a of the outer tube 2 in the direction of inside the branched part 6 is set to be 5 to 15 mm In other words, the opening 5a and the opening 7a are set so that the positions along the axis line of the inner tube 4 are separated in the range of 5 to 15 mm.

Further, in the catheter 1, in the branched part 6, the ratio of a cross-sectional area SB of the second lumen 7 formed within the inner tube 4 to a cross-sectional area SA of the first lumen 5 formed by at least the inside of the outer tube 2 and the outside of the inner tube 4 (which is acquired by dividing the cross-sectional area SA of the first lumen by the cross-sectional area SB of the second lumen) is set so as to become equal to or not smaller than that in the conduit part 2A.

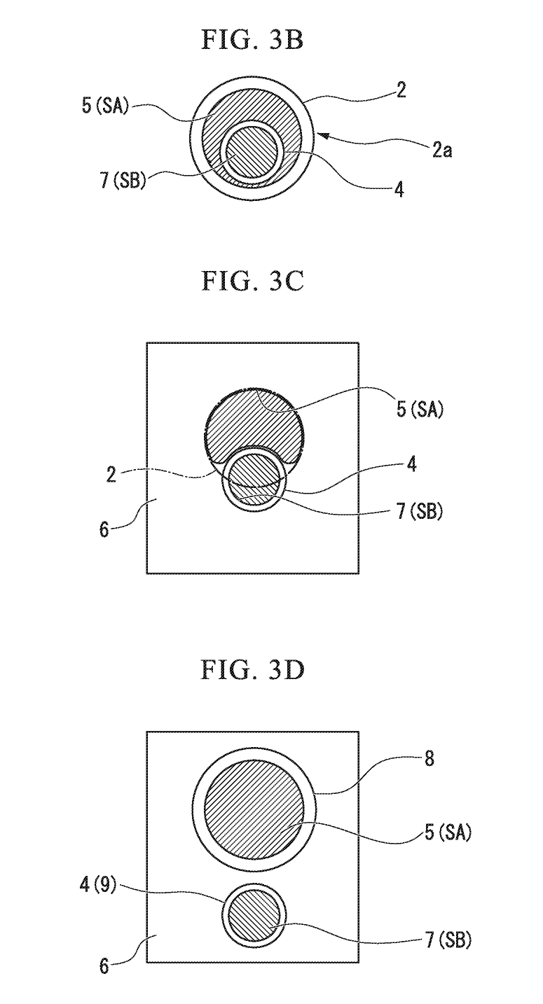

FIGS. 3A to 3D are arrow sectional views in the catheter of the present embodiment.

Specifically, at a position illustrated by arrow A-A in FIG. 1, as illustrated in FIG. 3A, the central axis of the outer tube 2 in the longitudinal direction and the central axis of the inner tube 4 in the longitudinal direction are in a coaxial state.

At a position illustrated in FIG. 2 by arrow B-B that is located in the vicinity of the outside of the branched part 6, as illustrated in FIG. 3B, the central axis of the outer tube 2 and the central axis of the inner tube 4 are in a state that is eccentric from the coaxial state (in a state where the central axis of the outer tube 2 and the central axis of the inner tube 4 are offset from each other).

At a position where the central axis of the outer tube 2 and the central axis of the inner tube 4 are separated from each other but are not branched, in the branched part 6 illustrated in FIG. 2 by arrow C-C, the inner tube 4, as illustrated in FIG. 3C, is in a state where the inner tube 4 protrudes from a sectional outline of the outer tube 2 illustrated by a one-dot chain line.

At a position inside the branched part 6 and closer to the base end of the branched part 6 illustrated by arrows D-D in FIG. 2, the outer tube 2 and the inner tube 4 are branched as illustrated in FIG. 3D, a tube 8 instead of the outer tube 2 is provided in the first lumen 5, and a tube 9 instead of the inner tube 4 is provided in the second lumen 7. Here, the internal diameter of the tube 8 is set to be equal to the internal diameter of the outer tube 2, and the internal diameter of the tube 9 is set to be equal to the internal diameter of the outer tube 2.

In this way, in the branched part 6 in the present embodiment, the length which the end 4a of the inner tube 4 extends from the end 2a of the outer tube 2 in the direction of inside the branched part 6 is set to be in a predetermined range. In other words, the opening 5a and the opening 7a are set so that the positions along the axis line of the inner tube 4 are separated within the range. Thereby, in the catheter 1, sealability of the first lumen 5 and the second lumen 7 in the branched part 6 can be easily maintained even if they are coaxial lumens. Simultaneously, manufacturing can be made easily without degrading sealability, cost of producing the catheter 1 is reduced, and it is possible to prevent a sheath from being crushed by ejection pressure at the time of outsert of the branched part 6.

Next, a process of manufacturing the catheter 1, especially, steps performed in the vicinity of the branched part 6 of the catheter 1 will be described.

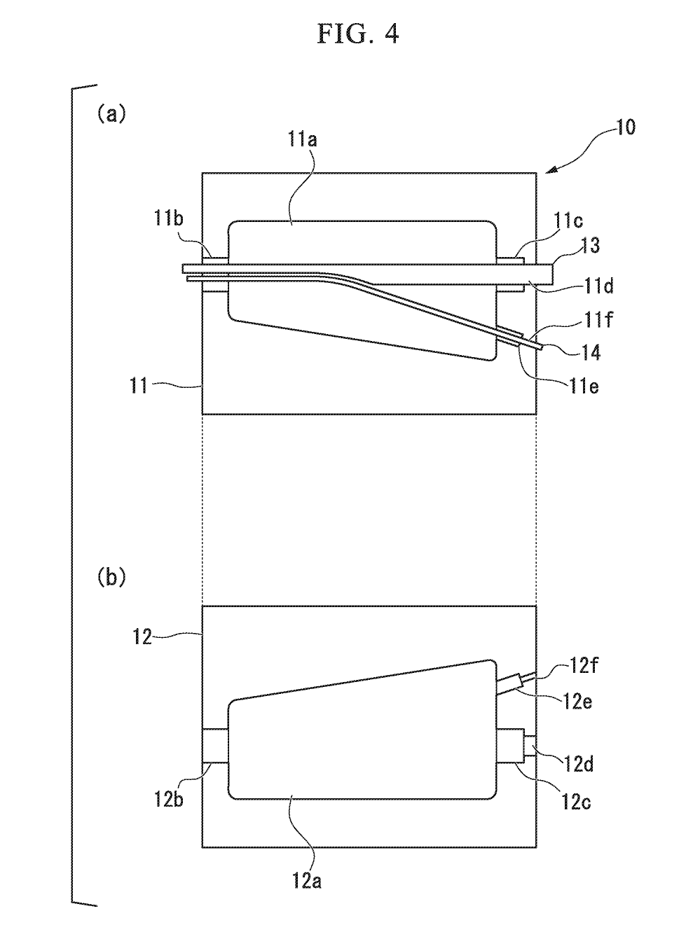

In the method for producing the catheter 1 of the present embodiment, a mold 10 as illustrated in FIG. 4 to FIG. 6 is used.

The mold 10, as illustrated in FIG. 4, is constituted of an upper mold (outer mold) 11, a lower mold (outer mold) 12, a thick core bar (first core bar) 13, and a core bar (second core bar) 14.

An inner surface (mating surface) of the upper mold 11 has a space part (cavity) 11a that constitutes a resin molding space serving as the branched part 6, a first groove 11b that allows the tip side of the outer tube 2 to be disposed therein, a second groove 11c that allows the base end side of the outer tube 2 to be placed therein, a third groove 11d that allows the thick core bar 13 to be placed therein continuously with the second groove 11c, a fourth groove 11e that allows the base end side of the inner tube 4 to be disposed therein, and a fifth groove 11f that allows the core bar 14 to be placed therein continuously with the fourth groove 11e.

The first to fifth grooves (groove parts) 11b to 11f are provided on a reference surface that is substantially the same plane at positions that faces each other with the space part 11a capable of forming the branched part 6 interposed therebetween.

A gate (not illustrated) for filling of resin constituting the branched part 6 is provided in the space part 11a so as to communicate with the space part 11a from a side surface part of the upper mold 11.

Although the space part 11a assumes, for example, a rectangular shape as the branched part 6, the space part just has to have a predetermined shape.

An inner surface (mating surface) of the lower mold 12 similarly has a space part (cavity) 12a that constitutes a resin molding space serving as the branched part 6, a first groove 12b that allows the tip side of the outer tube 2 to be disposed therein, a second groove 12c that allows the base end side of the outer tube 2 to be placed therein, a third groove 12d that allows the thick core bar 13 to be placed therein continuously with the second groove 12c, a fourth groove 12e that allows the base end side of the inner tube 4 to be disposed therein, and a fifth groove 12f that allows the core bar 14 to be placed therein continuously with the fourth groove 12e.

The first to fifth grooves (groove parts) 12b to 12f are provided on a reference surface that is substantially the same plane at positions that faces each other with the space part 12a capable of forming the branched part 6 interposed therebetween.

The first groove 11b or 12b allow the cavity 11a or 12a and the outside of the mold 11 or 12 to communicate with each other, and is set such that the inside of the first groove 11b or 12b is sealed by the outer tube 2 when the outer tube 2 is sandwiched between the molds 11 and 12, with a shape corresponding to the external diameter of the outer tube 2.

The second groove 11c or 12c extends to the outside of the mold 11 or 12 from the cavity 11a or 12a, is set so as to have a shape corresponding to the external diameter of the outer tube 2 or the tube 8, and is formed at a position to which the first groove 11b or 12b is extended. The second groove 11c or 12c similarly is set such that the inside of the second groove 11c or 12c is sealed by the outer tube 2 when the outer tube 2 is sandwiched between the molds 11 and 12, with a shape corresponding to the external diameter of the outer tube 2.

The third groove 11d or 12d is set so as to become coaxial with the second groove 11c or 12c, allows the second groove 11c or 12c and the outside of the mold 11 or 12 to communicate with each other, and is set such that the inside of the third groove 11d or 12d is sealed by the thick core bar 13 when the thick core bar 13 is sandwiched between the molds 11 and 12, with a shape corresponding to the external diameter of the thick core bar 13.

The fourth groove 11e or 12e extends from the cavity 11a or 12a to the outside of the mold 11 or 12, is set to have angles from the first groove 11b or 12b so as to become eccentric from a line that connects the first groove 11b or 12b and the second groove 11c or 12c to each other, and is set such that the inside of the fourth groove 11e or 12e is sealed by the inner tube 4 (9) when the inner tube 4 (9) is sandwiched between the molds 11 and 12, with a shape corresponding to the external diameter of the inner tube 4 (9).

The fifth groove 11f or 12f is set so as to become coaxial with the fourth groove 11e or 12e, allow the fourth groove 11e or 12e and the outside of the mold 11 or 12 to communicate with each other, and is set such that the inside of the fifth groove 11f or 12f is sealed by the core bar 14 when the core bar 14 is sandwiched between the molds 11 and 12, with a shape corresponding to the external diameter of the core bar 14.

The first groove 11b or 12b, the second groove 11c or 12c, and the fourth groove 11e or 12e are disposed so as to have a shape that branches into a Y shape.

In addition, the second groove 11c or 12c and the fourth groove 11e or 12e can be made to extend to the outside of the mold 11 and 12, and the third groove 11d or 12d and the fifth groove 11f or 12f cannot be provided.

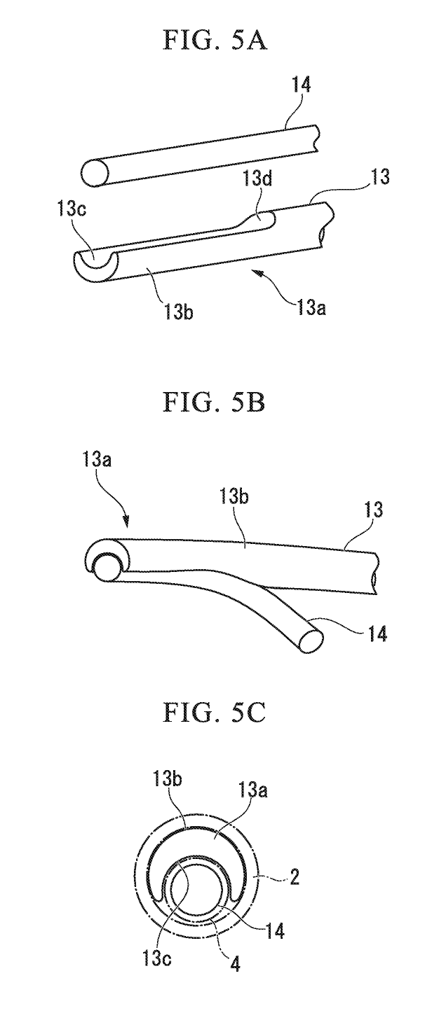

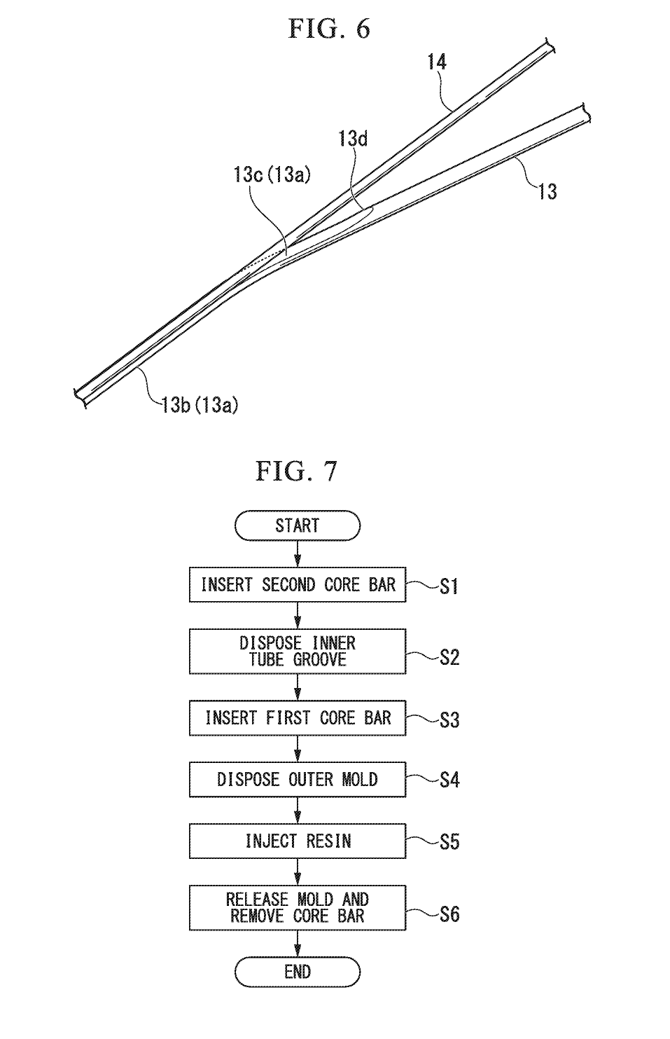

As illustrated in FIGS. 5A to 5C and FIG. 6, the thick core bar (first core bar) 13 has an insertion part 13a that is made to be insertable into a gap between the outer tube 2 and the inner tube 4 located within the outer tube 2 so as to form the first lumen 5 in the branched part 6. The insertion part 13a has an external diameter equal to the internal diameter of the outer tube 2, and has a substantially cylindrical outer surface part 13b, and an inner surface part 13c along the outside of the inner tube 4. The inner surface part 13c of the insertion part 13a is a groove 13c corresponding to the external form of the inner tube 4. At a position corresponding to the branched part 6, a termination end 13d is provided in the groove 13c of the insertion part 13a in a length direction thereof, and the thick core bar 13 increases in diameter from the termination end 13d toward a base end side of the branched part 6. That is, the termination end 13d is provided in the groove 13c such that the first lumen 5 increases in diameter. That is, a tip side of the thick core bar 13 serves as the insertion part 13a from the termination end 13d, and the groove 13c is provided over the entire length of the insertion part 13a.

The sectional shape of the insertion part 13a in a direction intersecting the axis is formed in a substantially crescent shape.

Additionally, the core bar (second core bar) 14 is made to be insertable into the inner tube 4 (9) and is formed in a columnar shape having the same outside dimension as the internal diameter of the inner tube 4, and the core bar 14 is set such that the inner tube 4 can be positioned inside the outer tube 2, in a state where the core bar 14 is inserted into the inner tube 4 when being disposed within the groove 13c.

The sectional shape of the core bar (second core bar) 14 in the direction intersecting the axis is substantially circular.

Although the material that constitutes the core bars 13 and 14 is not particularly limited if this material is not a material in which the core bars 13 and 14 are melted or deformed, metal is preferable. For example, stainless steel, iron, or the like is included.

Next, the method for producing the catheter 1 in the present embodiment will be described.

As illustrated in FIG. 7, the method for producing the catheter 1 in the present embodiment has a second core bar inserting step S1, an inner tube groove disposing step S2, a first core bar inserting step S3, an outer mold disposing step S4, a resin injecting step S5, and a mold releasing and core bar removing step S6.

In the second core bar inserting step S1, first, the second core bar 14 is inserted into the base end side of the inner tube 4. In this case, the insertion position of the second core bar 14 to the tip side with respect to the inner tube 4 is set as up to the outside position of the branched part 6 or the outside position of the mold 11 or 12.

In addition, in a case where the tube 9 or the like in which the inner tube 4 is a separate member on the base end side of the branched part 6 is used, the tube 9 that is the separate member is inserted into the second core bar 14 serving as the base end side of the branched part 6 at a predetermined position.

In the inner tube groove disposing step S2, the inner tube 4 into which the second core bar 14 is inserted in the second core bar inserting step S1 is disposed in the groove 13c of the first core bar 13 in a state where the inner tube 4 is folded corresponding to the termination end 13d of the groove 13c. In this case, the position of the inner tube 4 which has the second core bar 14 inserted thereinto and is separated from the groove 13c of the first core bar 13 is located inside the branched part 6 and need not strictly coincide with the termination end 13d, but the end 4a of the inner tube 4 is set to be separated from the end 13d for a distance of the above-described predetermined range.

Next, in the first core bar inserting step S3, the tip side of the second core bar 14, the inner tube 4 into which the second core bar 14 is inserted, and the insertion part 13a of the first core bar 13 in which the base end side of the inner tube 4 is disposed within the groove 13c are inserted from the termination end side of the outer tube 2.

Otherwise, as a step so far, the second core bar 14 may be inserted into the inner tube 4 inserted into the outer tube 2 in advance, and the insertion part 13a of the first core bar 13 may be inserted between the outer tube 2 and the inner tube 4.

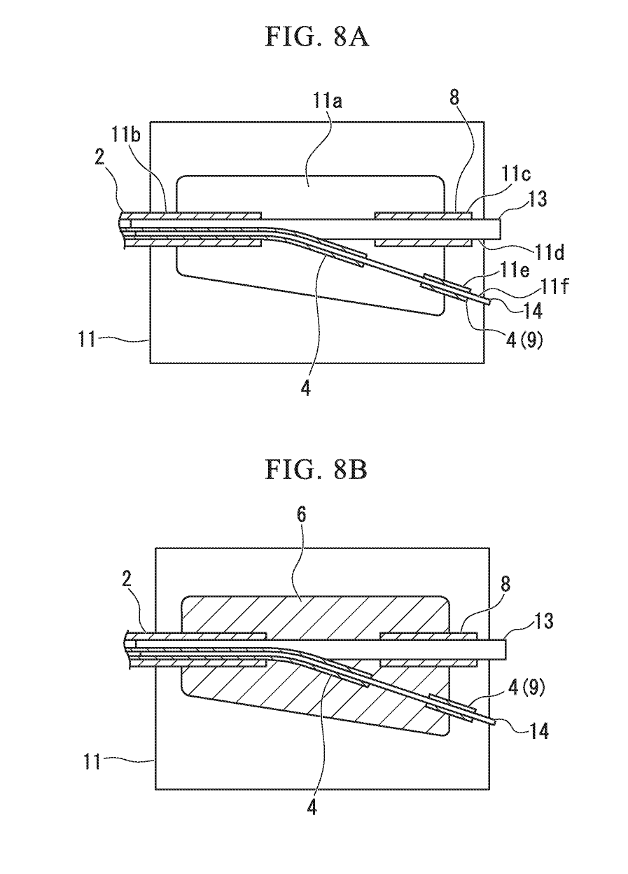

Next, as the outer mold disposing step S4, the second core bar 14, the inner tube 4, the first core bar 13, and the outer tube 2 are disposed in the corresponding groove parts 11b to 12f of the outer molds 11 and 12. Specifically, as illustrated in FIG. 8A, the position of the outer tube 2 is restricted by the first grooves 11b and 12b, the position of the tube 8 is restricted by the second grooves 11c and 12c, the position of the thick core bar 13 is restricted by the third grooves 11d and 12d, the position of the inner tube 4 or the position of the tube 9 is restricted by the fourth grooves 11e and 12e, and the position of the core bar 14 is restricted by the fifth grooves 11f and 12f. Additionally, the position of the second core bar 14 in the inserted state, the position of the inner tube 4, the position of the first core bar 13, and the position of the outer tube 2 are restricted by the first grooves 11b and 12b.

Accordingly, a state where the position of the second core bar 14 in the inserted state, the position of the inner tube 4, and the position of the first core bar 13, and the position of the outer tube 2 are restricted by the first groove 11b to 12f so as to have a shape branched in a Y shape is brought about.

Next, as illustrated in FIG. 8B, as the resin injecting step S5, the upper mold 11 and the lower mold 12 are made to abut against each other in the inner surfaces (mating surfaces) thereof, both ends thereof are fixed to each other, and molten resin is injected into the spaces parts 11a and 12a from the gate (not illustrated) to mold the branched part 6.

Although the temperature of a cylinder in a resin extrusion part when performing molding is not particularly limited because this temperature is dependent on the resin to be extruded, 190° C. to 230° C. is preferable, and 200° C. to 220° C. is particularly preferable. Additionally, although mold temperature is based on the shape of the branched part 6, 10° C. to 50° C. is preferable, and 15° C. to 20° C. is particularly preferable.

The injected resin forms the branched part 6 by the cavities 11a and 12a. In the branched part 6, the outer tube 2 and the inner tube 4 that are coaxial with each other is disposed on the tip side, and the tube 8 and the tube 9 serving as branched lumens are disposed on the base end side. Additionally, the tube 8 and the tube 9 are provided with an axis angle formed between each other within almost the same plane which the respective lumens form.

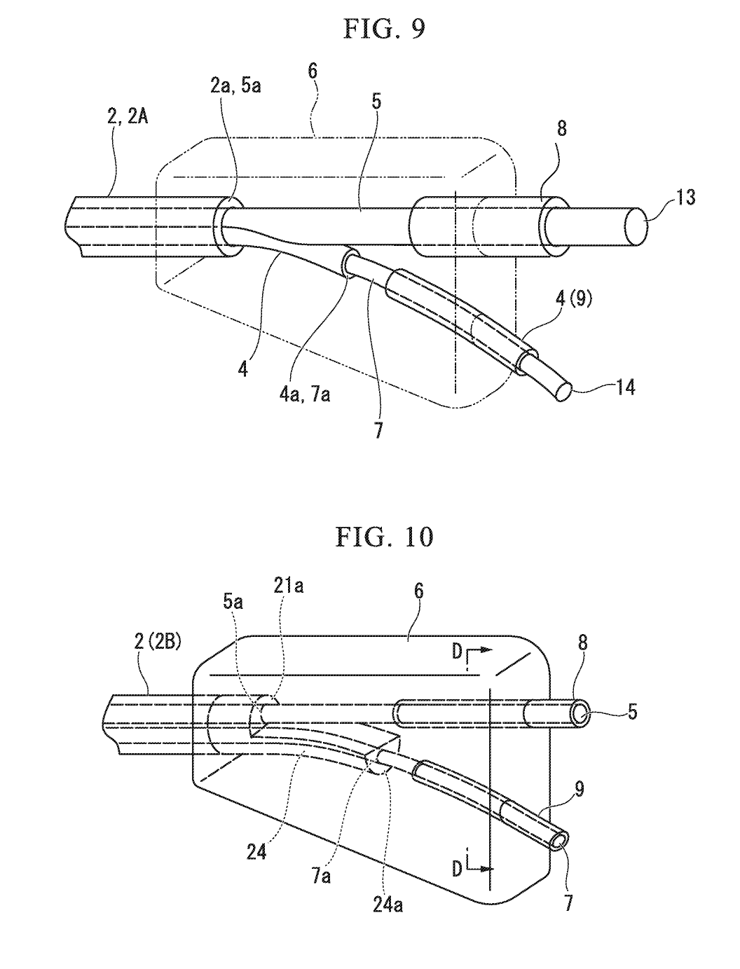

Then, as the mold releasing and core bar removing step S6, first, as illustrated in FIG. 9, the molds 11 and 12 are released. Then, the branched part 6 is formed by removing the core bars 13 and 14.

Moreover, if required, bonding or deposition is performed outside the branched part 6. Accordingly, the catheter 1 can eventually be obtained.

Although the catheter, the catheter production mold, and the catheter production method of the present invention have been described on the basis of the illustrated embodiment, the present invention is not limited to this, and the configurations of the respective parts can be substituted with arbitrary configurations that can exhibit the same functions.

For example, although a case where one inner tube 4 is provided has been described in the present embodiment, the present invention can be used even in a case where a plurality of (such as, two) inner tubes are provided.

Additionally, as the outer mold disposing step S4, as illustrated in FIG. 5B, the thick core bar 13 is substantially linear, and the core bar 14 is curved in the vicinity of the termination end 13d of the groove 13c. However, as illustrated in FIG. 6, the core bar 14 may be substantially linear, and the thick core bar 13 may be curved in the vicinity of the termination end 13d of the groove 13c.

A second embodiment of a catheter related to the present invention will be described referring to the drawings.

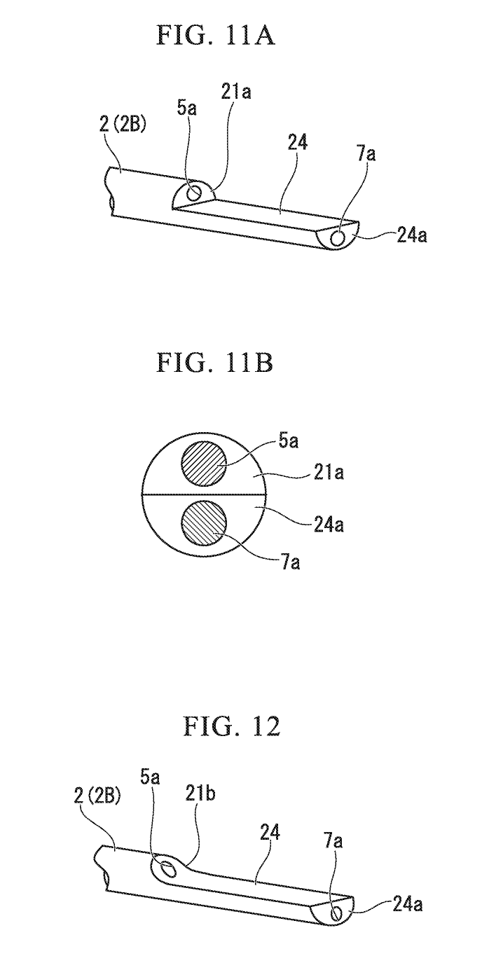

FIG. 10 is a perspective view illustrating the second embodiment of a catheter related to the present invention, and FIG. 11A and FIG. 11B are enlarged perspective views illustrating the conduit part inside the branched part 6. The difference between this embodiment and the first embodiment is the point related to the cross-sectional shape and the shape of the end of the conduit part 2B. In the following description, the components corresponding to the first embodiment are denoted with the same reference signs, and detailed description of the respective components will be omitted.

In the catheter of this embodiment, as shown in FIGS. 10, 11A and 11B, the first lumen 5 and the second lumen 7 are integrally formed as conduits with circular cross-section in the conduit part 2B.

Inside the branched part 6, as shown in FIGS. 10, 11A and 11B, the end face 21a where the opening 5a of the first lumen 5 is provided and the end face 24a where the opening 7a of the second lumen 7 is provided are provided at the end of the conduit part 2B. At the base end side of the conduit part 2B, the end face 21a and the end face 24a form a level difference.

The end face 21a and the end face 24a are parallel with each other, and are arranged to cross the axis line of the conduit part 2B at right angles.

The end face 21a of this embodiment corresponds to the end 2a of the outer tube 2 of the first embodiment, and the end face 24a of this embodiment corresponds to the end 4a of the inner tube 4 of the first embodiment. Similar to the first embodiment, the openings 5a, 7a are provided at the end faces 21a, 24a.

The end faces 21a and 24a at the base end side of the conduit part 2B are set so that the opening 5a of the first lumen 5 and the opening 7a of the second lumen 7 are disposed along the different axis line (in the direction of the channel) with each other, and a level difference is formed by the end face 21a and the end face 24a.

Specifically, as shown in FIG. 10 and FIG. 11A, the end face 21a of the conduit part 2B is arranged at the position of 5 to 10 mm from the left side end position of the branched part, and the end face 24a of the conduit part 2B is arranged at the position of 10 to 20 mm from the left side end position of the branched part 6.

That is, in the level difference of the conduit part 2B, the length which the end face 24a extends from the end face 21a in the direction of inside the branched part 6 is set to be 5 to 15 mm In other words, the opening 5a and the opening 7a are set so that the positions along the channel of the lumens 5 and 7 of the conduit part 2B are different with each other in the range of 5 to 15 mm.

In this embodiment, similar to the first embodiment described above, a cylindrical metal core such as the metal core 14 is inserted into a position of the first lumen or the second lumen and forms the branched part 6.

Also in this embodiment, the sealability of the first lumen 5 and the second lumen 7 in the branched part 6 is easily maintained. Furthermore, since the conduit part 2B can be manufactured only by cutting and removing the end of the conduit part 2B, which forms the first lumen 5 and the second lumen 7, so that the end faces 21a and 24a are provided, it becomes possible to reduce a process of operation, shorten a productive time, reduce a manufacturing cost, and maintain the sealability as in the first embodiment.

In addition, while the level difference is formed by the parallel end faces 21a and 24a in this embodiment, the level difference may be formed as the inclined end face 21b as shown in FIG. 12.

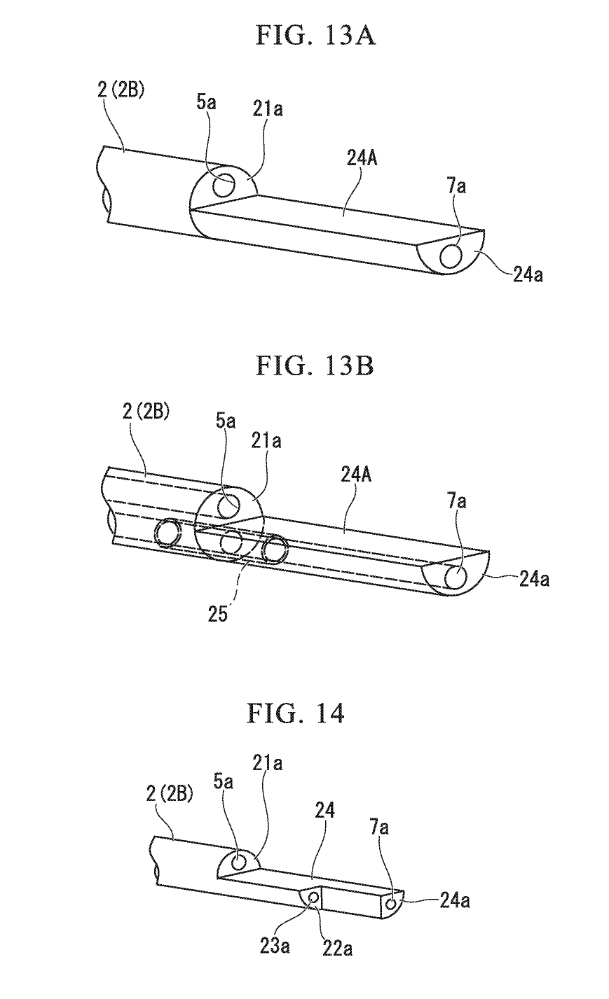

Further, in this embodiment, the end 24 of the inner tube may be extended integrally combining the end face 21 a with another component 24A as shown in FIG. 13A. In that case, as shown in FIG. 13B, the tube 25 may be provided at a joining part with the second lumen 7 to improve the sealability.

Moreover, when three or more lumens are formed, it becomes possible to increase the sealability in the branched part 6 in the catheter having a plurality of lumens by forming the level difference by the end faces 21a, 22a, and 24a. FIG. 14 shows an example having three lumens. In this case, the opening 5a, the opening 23a, and the opening 7a of each lumen are arranged to be in a position along different channels (axis lines) with each other. It is possible to be located at the end of the conduit part 2B where the level difference is formed by the end faces 21a, 22a, and 24a.

A third embodiment of a catheter related to the present invention will be described referring to the drawings.

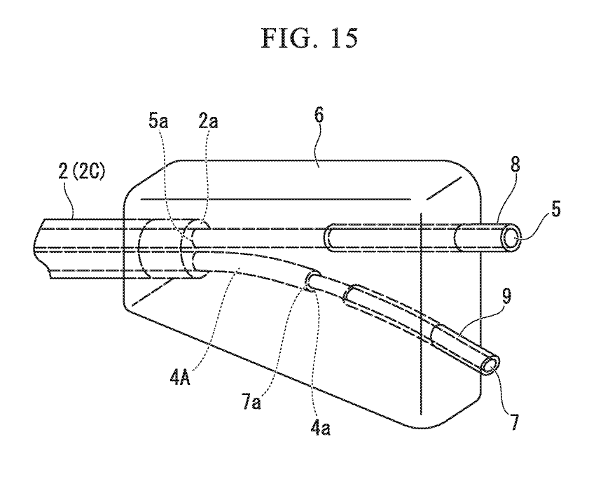

FIG. 15 is a perspective view illustrating the third embodiment of a catheter related to the present invention, and the difference between this embodiment and the first embodiment is the point related to the shape of the end of the conduit part 2C and an insertion tube 4. In the following description, the components corresponding to the second embodiment are denoted with the same reference signs, and detailed description of the respective components will be omitted.

In the conduit part 2C of this embodiment, the first lumen 5 and the second lumen 7 are formed integrally as conduits having an equal circular cross-section as in the conduit part 2B in the second embodiment, as shown in FIG. 15. Furthermore, the end 2a of the conduit part 2C in the branched part 6 is provided as the plane that is perpendicular with the axis line, and the opening 5a of the first lumen 5 is formed at the end thereof. In the second lumen 7, the insertion tube (inner tube) 4A is inserted into the channel only at the neighborhood of the branched part 6. The end face 4a inside the branched part 6 of the insertion tube 4A is arranged to be separated from the end 2a in the direction of the channel as the end 4a of the inner tube 4 of the first embodiment, and the opening 7a of the second lumen 7 is arranged to be separated from the opening 5a of the first lumen 5 for a predetermined range. That is, the opening 7a of the second lumen 7 is arranged by being extended by the insertion tube (inner tube) 4A towards the inner side of the branched part 6 from the opening 5a of the first lumen 5.

Specifically, as shown in FIG. 15, the end face 2a of the conduit part 2C is arranged at 5 to 10 mm separated from the left side end of the branched part 6, and the end face 4a of the insertion tube 4A is arranged at 10 to 20 mm separated from the left side end of the branched part 6.

That is, in the conduit part 2C, the length which the end face 4a is moved extending from the end face 2a in the direction inside the branched part 6 is set to be 5 to 15 mm. In other words, each of the opening 5a and the opening 7a is arranged at a different position in the range of 5 to 15 mm along the channel of the lumens 5 or 7 of the conduit part 2C. In the conduit part 2C, the end 2a and the end 4a form the level difference. Also, the insertion tube 4A is inserted toward the tip side of the conduit part 2C, which is left-hand side in the figure, from the end face 2a of the conduit part 2C for a depth size that is set as 5 to 20 mm.

In this embodiment, similar to the first and second embodiments described above, the branched part 6 is formed by inserting a cylindrical metal core such as the core bar 14 at a position where the first and second lumens are provided.

Also in this embodiment, the same effect can be attained as the first and second embodiments described above. Further, since the sealability in the branched part 6 can be presented enough only by inserting the insertion tube 4A after cutting the conduit part 2C as a plane, the workability in a manufacturing process is improved and it becomes possible to reduce a manufacturing cost.

What is claimed is:

1. A catheter comprising:
a conduit part having a plurality of lumens whose external diameter at a tip side thereof is constant, each of the plurality of lumens extending along an axis line inside the conduit part; and
a branched part that separates axis lines of the plurality of lumens from each other at a base end side of the conduit part,
wherein, at an end of the conduit part inserted into inside the branched part, an opening of each of the plurality of lumens is provided along a different axis line with each other, and a level difference is formed at the end of the conduit part.

2. The catheter according to claim 1, wherein
an inner tube in which at least one lumen of the plurality of lumens are formed is provided at inside of the conduit part, and
an end of the inner tube is extended toward a base end side of the branched part than the end of the conduit part inserted into inside the branched part.

3. The catheter according to claim 2, wherein
the inner tube is provided only around inside of the branched part.

4. The catheter according to claim 1, wherein
an inner tube in which at least one lumen of the plurality of lumens are formed is provided at inside of the conduit part,
the inner tube is provided as a coaxial lumen inside an outer tube of the conduit part, and
an end of the inner tube is extended toward a base end side of the branched part than an end of the outer tube inserted into inside the branched part.

5. A catheter production mold for producing a catheter including:
a conduit part having a plurality of lumens whose external diameter at a tip side thereof is constant, each of the plurality of lumens extending along an axis line inside the conduit part; and
a branched part that separates axis lines of the plurality of lumens from each other at a base end side of the conduit part, wherein
an inner tube is provided as the lumen at inside of the conduit part,
the inner tube is provided as a coaxial lumen inside an outer tube of the conduit part, and
an end of the inner tube is extended toward a base end side of the branched part than an end of the outer tube inserted into inside the branched part,
the mold comprising:
a first core bar inserted into a gap between the outer tube and the inner tube located within the outer tube;
a second core bar inserted into the inner tube; and
an outer mold that covers the first core bar and the second core bar to form an external form of the branched part,
wherein in the first core bar, an insertion part to be inserted into the outer tube has an outer surface part along inside of the outer tube, and an inner surface part along the outside of the inner tube, the inner surface part is a groove corresponding to an external form of the internal tube, and a termination end is provided in the groove of the insertion part in a length direction of the groove,
wherein the first core bar increases in diameter from the termination end toward a base end side of the branched part, and
wherein groove parts that allow the outer tube, the inner tube, and the first and second core bars inserted into the outer and inner tubes to be disposed therein and branches into a Y shape, and a space part that allows the branched part to be formed therein together with the groove parts are disposed in the outer mold.

6. The catheter production mold according to claim 5,
wherein a sectional shape of the insertion part of the first core bar in a direction intersecting an axis is substantially a crescent, and
wherein a sectional shape of the second core bar in the direction intersecting the axis is substantially circular.

7. A catheter production method for producing a catheter using the mold according to claim 5, the method comprising:
a step of inserting the second core bar into the inner tube;
a step of disposing the inner tube, into which the second core bar is inserted, in the groove of the first core bar;
a step of inserting the second core bar, the inner tube, and the first core bar into the outer tube;
a step of disposing the second core bar, the inner tube, the first core bar, and the outer tube in the corresponding groove parts of the outer mold;
a step of injecting resin into the space part to form the branched part; and
a step of releasing the mold and removing the core bars.
